(19) United States
(12) Patent Application Publication
(10) Pub. No.: US 2003/0157553 A1
(43) Pub. Date: Aug. 21, 2003

(54) Methods of assaying for G protein-coupled receptor ligands and modulators

(76) Inventor: Gabriel Berstein, Woburn, MA (US)

Correspondence Address: Intellectual Property Group, MILLENNIUM PHARMACEUTICALS, INC., 75 Sidney Street, Cambridge, MA, 02139, US

(21) Appl. No.: 09/800,137

(22) Filed: Mar. 5, 2001

Related U.S. Application Data
(60) Provisional application No. 60/186,706, filed on Mar. 3, 2000.

(51) Int. Cl. G01N 33/53

(52) U.S. Cl. 435/7.1

(57) ABSTRACT

The present invention features methods of assaying for ligands and/or modulators of G protein-coupled receptors, for example, orphan G protein-coupled receptors. The methods of the invention feature constitutively active arrestin mutants, in particular, phosphorylation independent mutants. Also described are specific phosphorylation independent arrestin mutants and methods of making such mutants.

### RELATED APPLICATIONS

This application claims the benefit of prior-filed provisional patent application U.S. Ser. No. 60/186,706, entitled “Methods of Assaying for G Protein-Coupled Receptor Ligands and Modulators”, filed Mar. 3, 2000 (pending). The content of the above referenced application is incorporated herein in its entirety by this reference.

### BACKGROUND OF THE INVENTION

G protein-coupled receptors (GPCRs) are integral membrane proteins involved in the transmission of signals from the extracellular environment to the cytoplasm (for review see e.g., Gutkind (1998) J. Biol. Chem. 273:1839-1842). The best known family of G protein-receptors (GPCRs), currently comprises more than 1000 members and exhibits a common structural motif consisting of seven membrane-spanning regions (Dohlman et al. (1987) Biochemistry 26:2657-2664). Sixteen distinct mammalian Gα protein subunits have been molecularly cloned and are divided into four families based upon sequence similarity: Gαs, Gαi, Gαq, and Gα₁₂. Similarly, eleven Gβ protein subunits and five G protein subunits have been identified. Thus, GPCRs are likely to represent the most diverse signal transduction systems in eukaryotic cells.

A diverse array of external stimuli including neurotransmitters, hormones, phospholipids, photons, odorants, certain taste ligands, and growth factors can activate members of this receptor family and promote interaction between the receptor and a G protein located on the intracellular side of the membrane. This causes the exchange of GDP for GTP bound to the G protein α subunit and further causes the dissociation of the βγ heterodimers. In turn, GTP-bound G protein α subunits or βγ complexes initiate intracellular signaling responses by acting on effector molecules such as adenylate cyclases or phospholipases or directly regulating ion channel or kinase function.

Signaling by diverse GPCR agonists is believed to be terminated by a uniform two-step mechanism (Freedman and Lefkowitz (1996) Recent Prog. Horm. Res. 51:319-353). According to the model, activated receptor is first phosphorylated by a G protein-coupled receptor kinase (GRK). An arrestin protein then binds to the activated phosphoreceptor, thereby blocking G protein interaction. Arrestin-receptor complex is subsequently internalized, whereupon receptor is either dephosphorylated and recycled back to the plasma membrane (resensitization) or sorted to lysosomes and destroyed (down-regulation). Thus, the formation of the arrestin-receptor complex appears to be the final step of desensitization and the first step of resensitization and/or receptor down-regulation.

GPCRs are activated by an extremely wide variety of external stimuli and are now believed to play a role in regulating the activity of virtually all eukaryotic cells. In contrast, the repertoire of receptor kinases and arrestins involved in the desensitization of these receptors is rather limited: six GRKs and four arrestins have thus far been found in mammals (reviewed in Freedman and Lefkowitz, supra).

### SUMMARY OF THE INVENTION

The fact that a limited number of receptor kinases and arrestins can regulate desensitization of numerous GPCRs makes these molecules attractive targets for research designed to identify potential modulators of GPCR signaling, in particular modulators of such receptors for use as human therapeutics. The present invention is based, at least in part, on a new use for phosphorylation-independent mutants of arrestin proteins as assay components in screening assays designed to identify ligands and/or modulators of G protein-coupled receptors (GPCRs). In particular, it has been discovered that phosphorylation-independent mutants of arrestin capable of binding GPCRs in a manner independent of the natural requirement for phosphorylation by intracellular receptor kinases. Such phosphorylation-independent mutants of arrestin retain there dependence on agonist for GPCR-binding, however. As such, phosphorylation-independent arrestin mutants are particularly well-suited for in vitro assays, for example, in assays that are directed to the identification of natural and surrogate agonists of orphan GPCRs (e.g., of known and/or orphan GPCRs, for example, in orphan GPCR ligand fishing assays). The phosphorylation-independent mutants of arrestin are also particularly well-suited for in vitro assays that are directed to the identification of GPCR antagonists and/or agonists.

In an exemplary embodiment, the present invention features methods of identifying a GPCR ligands which include contacting a composition containing the GPCR (e.g., a GPCR-containing membrane preparation) and a phosphorylation-independent arrestin mutant with a test compound and determining the ability of the test compound to modulate binding of the arrestin mutant to the GPCR, wherein modulation of binding indicates that the test compound is a GPCR ligand. In one embodiment, the GPCRs being assayed are known GPCRs. In another embodiment, the GPCRs being assayed are orphan G protein-coupled receptors. In another embodiment, the compound being identified is a GPCR modulator (e.g., an agonist or an antogonist or an inverse agonist for the GPCR being assayed). In another embodiment, the compound being identified is a natural and/or surrogate ligand for an orphan GPCR being assayed.

In another embodiment, the present invention features methods of identifying G protein-coupled receptor (GPCR) agonists which include contacting a composition containing the GPCR (e.g., a GPCR-containing membrane preparation), a phosphorylation-independent arrestin mutant and a test compound and determining the ability of the test compound to modulate binding of the arrestin mutant to the GPCR, wherein modulation of binding indicates that the test compound is a GPCR agonist.

In another embodiment, the present invention features methods of identifying G protein-coupled receptor (GPCR) antagonists which include contacting a composition containing the GPCR (e.g., a GPCR-containing membrane preparation), a phosphorylation-independent arrestin mutant and a GPCR agonist with a test compound and determining the ability of the test compound to modulate binding of the arrestin mutant to the GPCR, wherein modulation of binding indicates that the test compound is a GPCR antagonist.

The assays of the present invention are also applicable to screening pluralities of test compounds (e.g., a small molecule library of compounds) as well as screening composition which include more than one GPCR or a plurality of GPCRs. Test compounds can be included in aqueous or organic solutions. Arrestin mutants can be conjugated to a detectable marker (e.g., a colorometric label, a fluorescent label, a radioisotopic or radiolabel, or a reporter protein). Determining the ability of the test compound to modulate binding of the arrestin mutant to the GPCR can include detecting the binding of a labeled mutant to a gpcr (e.g., a GPCR in a membrane preparation). Determining the ability of the test compound to modulate binding of the arrestin mutant to the GPCR can include detecting a change in surface plasmon resonance (SPR).

Also featured are phosphorylation-independent arrestin mutants. Exemplary arrestin mutants include β-arrestin mutants (e.g., β-arrestin 1A mutants, β-arrestin 1B mutants and/or β-arrestin 2 mutants), arrestin C-mutants, X-arrestin mutants and/or visual arrestin mutants. Preferred phosphorylation-independent arrestin mutants have a mutation in the phosphorylation recognition region (e.g., have a mutation in an amino acid corresponding to any one of Lys 161, Arg 162, Arg 166, Arg170 and Lys 171 of SEQ ID NO: 4). Additional preferred constitutively-active mutants have a truncated C-terminal regulatory region.

### BRIEF DESCRIPTION OF THE DRAWINGS

FIG. 1 is a graphic depiction of the results of direct binding assays using various forms of arrestins and mutant arrestins (β-arrestins and/or visual arrestins) demonstrating that constitutively-active (i.e., phosphorylation-independent) mutants of the present invention bind G protein-coupled receptors (GPCRs) in a phosphorylation-independent manner. *, p<0.01, Student's t test, compared with the binding of corresponding wild type arrestin. FIG. 1A depicts the binding of arrestins and mutant arrestins to β₂-adrenergic receptors (β₂ARs) and FIG. 1B depicts the binding to light-activated rhodopsin (Rh).

FIG. 2 is a graphic representation of the results of agonist affinity shift assays designed to demonstrate the ability of various phosphorylation-independent arrestin mutants to form high agonist affinity complexes (HACs) with both phosphorylated and non-phosphorylated GPCRs. Unphosphorylated (FIG. 2A) or phosphorylated (FIG. 2B) β₂ARs were incubated with [¹²⁵I] iodopindolol (IPIN) and the indicated concentration of agonist in the absence (∘) or presence of 1 μM β-arrestin (●), β-arrestin-(Arg169→Glu) (▾), β-arrestin-(1-382) (▪), or β-arrestin-(1-393) (♦) as described in Example 2. Means±S.D. are presented.

FIG. 3 is a graphic depiction of the molecular architecture of arrestins and sequence homology of the phosphorylation recognition region. The major functional regions are designated as follows: R1, basic N-terminal regulatory region; R2, acidic C-terminal regulatory region; A, activation-recognition region; P, phosphorylation recognition region; S, secondary binding site region. The residues corresponding to the borders between the functional regions are shown below the schematic (numbering as in bovine β-arrestin 1A, GenBank™ Accession No. P17870, SEQ ID NO: 1). Sequences of putative phosphorylation-recognition regions of bovine β-arrestin 1A (corresponding to amino acids 155-184 of SEQ ID NO: 1), human β-arrestin 1A (corresponding to amino acids 155-184 of SEQ ID NO: 2), human β-arrestin 1B (corresponding to amino acids 155-184 of SEQ ID NO:3), human β-arrestin 2 (corresponding to amino acids 156-185 of SEQ ID NO: 4), human arrestin C (corresponding to amino acids 151-181 of SEQ ID NO: 5), and human X arrestin (corresponding to amino acids 152-181 of SEQ ID NO: 6) are indicated below the schematic.

FIG. 4A-B is a Clustal W alignment of the amino acid sequences of the following human arrestin proteins: β-arrestin 1A (GenBank™ Accession No. AAC33295, corresponding to SEQ ID NO: 2), β-arrestin 1B (GenBank™ Accession No. AAC34123, corresponding to SEQ ID NO: 3), β-arrestin 2 (GenBank™ Accession No. AAC99468, corresponding to SEQ ID NO: 4), arrestin C (GenBank™ Accession No. AAC78395, corresponding to SEQ ID NO: 5), and X arrestin (GenBank™ Accession No. AAB84302, corresponding to SEQ ID NO: 6). The alignment was generated using the Clustal W1.7 algorithm (parameters are as follows: Pairwise Alignment, K-tuple=1, Window size=5, Gap Penalty=3, Number of Top Diagonals=5; Multiple Alignment Parameters, Gap Open Penalty=10, Gap Extension Penalty=0.05, Blosum Matrix). Phosphorylation recognition domains are indicated in bold and C-terminal regulatory domains are indicated in bold italics. Asterisks above the β-arrestin 1A amino acid sequence indicate exemplary resudues mutated in phosphorylation-independent arrestin mutants of the present invention. Asterisks and (:) below the X arrestin amino acid sequence indicate conserved amino acids between arrestin proteins.

### DETAILED DESCRIPTION OF THE INVENTION

The present invention features phosphorylation-independent arrestin mutants and, in particular, features use of such mutants in in vitro screening assays.

### General Overview of Assay

In one embodiment, the present invention features methods of identifying a GPCR ligands which include contacting a composition containing the GPCR (e.g., a GPCR-containing membrane preparation) and a phosphorylation-independent arrestin mutant with a test compound and determining the ability of the test compound to modulate binding of the arrestin mutant to the GPCR, wherein modulation of binding indicates that the test compound is a GPCR ligand. As used herein, the term “contacting” (i.e., contacting a composition comprising a G protein-coupled receptor with a compound or agent) is intended to include incubating the compound or agent and composition together in vitro (e.g., adding the compound or agent to the composition in a suitable vessel) such that the compound or agent has the potential, for example, to interact with and/or bind to a GPCR which exists in the composition.

In one embodiment, the GPCRs being assayed are known G protein-coupled receptors and the ligand being identified is an unknown, additional or alternative ligand for the GPCR being assayed. In another embodiment, the GPCRs being assayed are orphan G protein-coupled receptors and the ligand being identified is a natural ligand for the orphan GPCR being assayed or, alternatively, is a surrogate ligand for the orphan GPCR being assayed.

In another embodiment, the present invention features methods of identifying a G protein-coupled receptor (GPCR) agonists which include contacting a composition containing the GPCR (e.g., a GPCR-containing membrane preparation) and a phosphorylation-independent arrestin mutant with a test compound and determining the ability of the test compound to modulate binding of the arrestin mutant to the GPCR, wherein modulation of binding indicates that the test compound is a GPCR agonist.

In another embodiment, the present invention features methods of identifying a G protein-coupled receptor (GPCR) antagonists which include contacting a composition containing the GPCR (e.g., a GPCR-containing membrane preparation), a phosphorylation-independent arrestin mutant and a GPCR agonist with a test compound and determining the ability of the test compound to modulate binding of the arrestin mutant to the GPCR, wherein modulation of binding indicates that the test compound is a GPCR antagonist.

Yet another embodiment of the present invention features a method for identifying a GPCR modulator which includes contacting a composition containing a constitutively-active mutant GPCR (CAM GPCR) and a phosphorylation-independent arrestin mutant with a test compound and determining the ability of the test compound to modulate binding of the arrestin mutant to the CAM GPCR, wherein modulation of binding indicates that the test compound is a GPCR modulator. As defined herein, a “constitutively active mutant GPCR” is a mutant GPCR protein that can activate GPCR-signaling activity in the absence of GPCR ligand or agonist binding. “Constitutively active mutant GPCRs” have basal receptor activity even in the absence of ligand binding. Accordingly, such “constitutively active mutant GPCRs” can be used in conjunction with the phosphorylation-independent mutant arrestins of the present invention is assay methods to identify GPCR modulators (e.g., inverse agonists of said GPCRs), even in the absence of GPCR ligands. In an exemplary embodiment, the invention features a method of identifying an orphan GPCR modulator (e.g., an inverse agonist of an orphan GPCR) which includes contacting an orphan CAM GPCR and a phosphorylation-independent arrestin mutant with a test compound and determining the ability of the test compound to modulate binding of the arrestin mutant to the orphan CAM GPCR, wherein modulation of binding indicates that the test compound is an orphan GPCR modulator.

In another embodiment, the present invention features methods of identifying a GPCR ligands which include contacting a composition containing the GPCR (e.g., a GPCR-containing membrane preparation) and a phosphorylation-independent arrestin mutant with a test compound and determining the ability of the test compound to modulate binding of the arrestin mutant to the GPCR, wherein modulation of binding indicates that the test compound is a GPCR ligand. In one embodiment, the GPCRs being assayed are known GPCRs. In another embodiment, the GPCRs being assayed are orphan G protein-coupled receptors. In another embodiment, the compound being identified is a GPCR modulator (e.g., an agonist or an antagonist or an inverse agonist for the GPCR being assayed). In another embodiment, the compound being identified is a natural and/or surrogate ligand for an orphan GPCR being assayed.

Determining the ability of a test compound to modulate binding of an arrestin mutant to a GPCR can include detecting a chance in the binding of a labelled mutant to the GPCR (e.g., the GPCR included in a membrane preparation). Determining the ability of the test compound to modulate binding of the arrestin mutant to the GPCR can also include detecting a change in surface plasmon resonance (SPR).

The assays of the present invention are applicable to screening pluralities of test compounds (e.g., a small molecule library of compounds) as well as screening composition which include more than one GPCR or a plurality of GPCRs. Test compound can be included in aqueous or organic solutions. Arrestin mutants can be conjugated to a detectable marker (e.g., a colorometric label, a fluorescent label, a radioisotopic or radiolabel, or a reporter protein).

Also featured are phosphorylation-independent arrestin mutants. Exemplary arrestin mutants include β-arrestin mutants (e.g., β-arrestin 1A mutants, β-arrestin 1B mutants and/or β-arrestin 2 mutants), arrestin C-mutants, X-arrestin mutants and/or visual arrestin mutants. Additional exemplary phosphorylation-independent arrestin mutants have a mutation in the phosphorylation recognition region (e.g., have a mutation in an amino acid corresponding to any one of Lys 161, Arg 162, Arg 166, Arg170 and Lys 171 of SEQ ID NO: 4). Additional preferred phosphorylation-independent arrestin mutants have a truncated C-terminal regulatory region. In an exemplary embodiment, a phosphorylation-independent arrestin mutant has the amino acid sequence set forth as SEQ ID NO: 7. In another embodiment, a phosphorylation-independent arrestin mutant has the amino acid sequence of SEQ ID NO: 2, lacking about amino acid residues 351-418. In another embodiment, a phosphorylation-independent arrestin mutant has the amino acid sequence set forth as SEQ ID NO: 8. In another embodiment, a phosphorylation-independent arrestin mutant has the amino acid sequence of SEQ ID NO: 4, lacking about amino acid residues 343-410. In another embodiment, a phosphorylation-independent arrestin mutant has the amino acid sequence of SEQ ID NO: 4, lacking about amino acid residues 344-409. In another embodiment, a phosphorylation-independent arrestin mutant has the amino acid sequence of SEQ ID NO: 5, lacking about amino acid residues 348-388. In yet another embodiment, a phosphorylation-independent arrestin mutant has the amino acid sequence of SEQ ID NO: 6, lacking about amino acid residues 348-388.

### II. Assay Formats

The present invention features a variety of assay formats suitable for identification of GPCR ligands and/or modulators. For example, assay components can be interacted or contacted in a reaction vessel suitable for reacting assay components (e.g., in a tube, for example, a test tube or microfuge tube, in a well, for example, in a microtitre-plate well, on a solid surface, for example, in a droplet or microdroplet, on a chip, or the like). In a preferred embodiment, at least one assay component can be immobilized in an assay well or on an assay surface either directly or indirectly and additional assay components interacted or contracted with said immobilized components.

In instances where, for example, it is desirable to utilize labeled or unlabeled arrestin mutants as an assay component or reagent, it may be desirable to immobilize the GPCR (or a GPCR-containing composition) to an assay surface (e.g., an assay well or chip) for example, in high throughput screening (HTS) or BIACORE™ assay formats described in Examples 4 and 5 herein.

Accordingly, in one embodiment, a GPCR or GPCR-containing composition (e.g., a purified and/or isolated GPCR in a suitable buffer or medium) can be immobilized to an assay surface directly, for example, via a covalent attachment (e.g., immobilization via native —NH₂, —SH, —CHO and/or COOH groups in the GPCR). In another embodiment, the composition can be immobilized to the assay surface indirectly. For example, an antibody specific for the GPCR can be immobilized on the assay surface (e.g., via direct conjugation or via an anti-Ig or anti-Fc antibody to the specific GPCR antibody. Alternatively, the GPCR (or GPCR-containing composition can be chemically modified (e.g., biotinylated) and immobilized, for example, on a streptaviden-coated assay surface.

The present invention further features assay formats in which GPCRs are included in membrane-like environment (e.g., membrane preparations or liposome) designed to allow interaction of the GPCRs with other assay components or reagents (e.g., mutant arrestin, and/or test compounds) in, for example, aqueous buffers.

In one embodiment, a GPCR in a membrane-like environment is immobilized to the assay surface directly. For example, a membrane prep (comprising at least one GPCR) can be immobilized via direct hydrophobic adsorption to the surface (e.g., via hydrophobic adsorption to an assay well using a hydrophobic adsorption (HPA) sensor chip). In another embodiment, a GPCR in membrane-like environment (e.g., in a membrane preparation comprising at least one GPCR) is immobilized to the assay surface indirectly. For example, an antibody specific for a target component in the composition can be immobilized on the assay surface e.g. via an anti-Fc or anti-IgG antibody to said component-specific antibody followed by immobilization of the GPCR-containing composition via interaction of said component-specific antibody with the target component included in the component included in the GPCR-containing composition. As used herein, a “target component” includes a component included, for example, in a membrane preparation to facilitate immobilization of the membrane preparation. Preferred target components include at least one GPCR in the membrane composition or a molecule (e.g., membrane protein) for which a binding partner is known, said binding partner being suitable for direct immobilization to the assay surface (e.g., an antibody specific for the target component, strepavidin, glutathione S-transferase (GST), maltose E binding protein, protein A, or the like). In yet another embodiment, for example, a GPCR in a membrane-like environment, can be immobilized to the assay surface via affinity capture of a second component (e.g., a target component) in the membrane preparation or liposome. For example, a known ligand for a first target receptor in the membrane preparation or liposome can be immobilized on the assay surface and used to affinity capture the membrane preparation or liposome, containing another or second receptor (e.g., an orphan GPCR for which identification of a surrogate ligand is sought). The above-described immobilization techniques are also applicable in instances in which it is desirable to utilize unlabeled arrestin mutants and/or unlabeled GPCRs as assay reagents or components, for example, in a BIACORE™ assay format as described herein. (See e.g Example 4.)

In yet other embodiments, it may be desirable to utilize unlabeled arrestin mutants immobilized on an assay surface (e.g, on a biomolecular sensor chip or BIACORE™ chip). Arrestin mutants can be immobilized using any of the direct or indirect immobilization techniques described above. Alternatively, arrestin mutants (or GPCRs, for example, in any of the above-described assay formnats) can be immobilized using any other art recognized means for immobilizing proteins to solid surfaces.

### III. Assay Reagents

A. Preparation of Mutant Arrestin Proteins

The present invention further features isolated or purified phosphorylation-independent arrestin mutants, or biologically active portions thereof. As used herein, a “phosphorylation-independent arrestin mutant” is an arrestin mutant that binds one or more GPCRs in a phosphorylation independent manner. A “phosphorylation-independent arrestin mutant” can be identified according to any assay designed to test the binding of the mutant to a GPCR. Mutants which have the ability to bind GPCR in the absence of phosphorylation of the GPCR (e.g., phosphorylation of the receptor by a GPCR-kinase or GRK) are said to bind in a “phosphorylation-independent” manner. An exemplary assay in which to test whether a potential phosphorylation-independent arrestin mutant has the ability to bind GPCR in a phosphorylation-independent manner is described in detail in Example 1.

The phrase “mutant arrestin” includes a protein differing in its amino acid sequence from a naturally-occurring arrestin protein such that the mutant arrestin has an altered biological activity as compared to the naturally-occurring arrestin protein. Exemplary “phosphorylation-independent arrestin mutants” include an arrestin proteins having a mutation in their amino acid sequence whish results in the arrestin being capable of binding a GPCR in the absence of phosphorylation of the arrestin protein. Additional exemplary “phosphorylation-independent arrestin mutants” include an arrestin protein having a mutation in its amino acid sequence which results in the arrestin protein having a substantially reduced dependence on phosphorylation as a requisite or requirement for GPCR-binding activity.

The phrase “mutant arrestin” is not intended to include arrestin proteins having amino acid substitutions, insertions and/or deletions which do not alter a biological activity of the protein. According to the present invention, arrestin protein having insertions, substitutions and/or deletions which do not affect or alter a biological activity of an arrestin can be termed “variants” or “homologues”. As such, the present invention is intended to include mutants (e.g., phosphorylation-independent mutants) of the arrestins set forth, for example, as SEQ ID NOs:2-5, as well as phosphorylation-independent mutants or other arrestin homologues and/or variants.

In one embodiment, an phosphorylation-independent arrestin mutant has an amino acid sequence at least 50%, 60%, 70%, 75%, 80%, 85%, 90%, 95% or more homologous or identical to any of the amino acid sequences set forth as SEQ ID NO.:2-5 and has a mutation in said amino acid sequence such that the mutant binds GPCRs in a phosphorylation-independent manner. To determine the percent homology of two amino acid sequences, the sequences are aligned for optimal comparison purposes (e.g., gaps can be introduced in the sequence of a first amino acid for optimal alignment with a second amino or nucleic acid sequence and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues at corresponding amino acid positions are then compared. When a position in the first sequence is occupied by the same amino acid residue as the corresponding position in the second sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100).

The comparison of sequences and determination of percent homology between two sequences can be accomplished using a mathematical algorithm. A preferred, non-limiting example of a mathematical algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov.

Another preferred, non-limiting example of a mathematical algorithm utilized for the comparison of sequences is the algorithm of Myers and Miller (1988) Comput. Appl. Biosci. 4:11-17. Such an algorithm is incorporated into the ALIGN program available, for example, at the GENESTREAM network server, IGH Montpellier, FRANCE (http://vega.igh.cnrs.fr) or at the ISREC server (http://www.ch.embnet.org). When utilizing the ALIGN program for comparing amino acid sequences, a PAM120 weight residue table, a gap length penalty of 12, and a gap penalty of 4 can be used.

For example, arrestin mutant proteins having amino acid substitutions at “non-essential” amino acid residues can be used according to the present invention. A “non-essential” amino acid residue is a residue that can be altered from the wild-type sequence of the exemplary arrestin mutant proteins described herein without altering the functional activity, whereas an “essential” amino acid residue is required for biological activity. For example, amino acid residues that are conserved among the arrestin mutant proteins of the present invention, are predicted to be particularly unamenable to alteration. Such conserved amino acids can be determined, at least for example, from an alignment of the amino acid sequences of the exemplary arrestin mutant proteins described herein.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Exemplary phosphorylation-independent arrestin mutants have a mutation in the phosphorylation recognition domain of the protein. As used herein, a “phosphorylation recognition domain” includes an arrestin protein domain having about 25-25, 26-34, 27-33, 28-32, 29-31 or 30 amino acid residues and is involved in regognition of phosphorylated GPCR by an arrestin protein. A phosphorylation recognition includes basic residues which are involved in recognition of phosphorylated receptor by the arrestin protein. In one embodiment, a “phosphorylation recognition domain” includes about amino acid residues 155-184 of SEQ ID NO: 2. In another embodiment, a “phosphorylation recognition domain” includes about amino acid residues 155-184 of SEQ ID NO: 3. In another embodiment, a “phosphorylation recognition domain” includes about amino acid residues 156-185 of SEQ ID NO: 4. In another embodiment, a “phosphorylation recognition domain” includes about amino acid residues 151-181 of SEQ ID NO: 5. In another embodiment, a “phosphorylation recognition domain” includes about amino acid residues 152-181 of SEQ ID NO: 6. Mutants within a “phosphorylation recognition domain” can include mutations of at least one basic residue involved in phosphoamino acid recognition of phosphorylated residues in a GPCR. Exemplary basic residues which can be mutated include lysines and/or arginines. In one embodiment, at least one amino acid residue selected from the group consisting of Lys 161, Arg 162, Arg 166, Arg170 and Lys 171 of SEQ ID NO: 4 is mutated. In another embodiment, a corresponding amino acid in an arrestin protein other than that set forth as SEQ ID NO: 4 (e.g., an amino acid corresponding to an amino acid residue selected from the group consisting of Lys 161, Arg 162, Arg 166, Arg170 and Lys 171 of SEQ ID NO: 4) is mutated in said other arrestin protein. Amino acid residues, for example, corresponding to an amino acid residue selected from the group consisting of Lys 161, Arg 162, Arg 166, Arg170 and Lys 171 of SEQ ID NO: 4, are set forth in FIG. 5. “Corresponding” amino acid residues in any other arrestin protein can be determined by optimally aligning the other arrestin to any one of the arrestin proteins set forth as SEQ ID NO:s 2-6 (e.g., the protein set forth as SEQ ID NO: 4) and visualizing basic amino acid residues within the other arrestin protein which align, for example, an amino acid residue selected from the group consisting of Lys 161, Arg 162, Arg 166, Arg170 and Lys 171 of SEQ ID NO: 4.

Exemplary phosphorylation-independent arrestin mutants alternatively or further have a truncation of a C-terminal regulatory domain of the protein. As used herein, a “C-terminal regulatory domain” includes an arrestin protein domain having about 40-70, preferably about 40-45, 45-50, 55-60 or 65-70 amino acid residues and serves a regulatory role in controlling arrestin binding to a phosphorylated (and/or) activated form of a GPCR without directly participating in the receptor interaction. In one embodiment, a C-terminally truncated arrestin mutant lacks at least about 10-20 C-terminal amino acid residues. In another embodiment, a C-terminally truncated arrestin mutant lacks at least about 20-30 C-terminal amino acid residues. In another embodiment, a C-terminally truncated arrestin mutant lacks at least about 30-40, 40-50, 50-60, 60-70, 70-80 or more C-terminal amino acid residues. Exemplary C-terminally truncated arrestin mutants inchlde the amino acid sequence of SEQ ID NO: 2, lacking about amino acid residues 351-418, the amino acid sequence of SEQ ID NO: 4, lacking about amino acid residues 343-410, the amino acid sequence of SEQ ID NO: 4, lacking about amino acid residues 344-409, the amino acid sequence of SEQ ID NO: 5, lacking about amino acid residues 348-388, or the amino acid sequence of SEQ ID NO: 6, lacking about amino acid residues 348-388.

In other exemplary embodiments, an arrestin mutant protein of the present invention is a mutant of a human arrestin protein selected from the group consisting of human β-arrestin 1A (GenBank™ Accession No. AAC33295, corresponding to SEQ ID NO: 2), human ™ -arrestin 1B (GenBank™ Accession No. AAC34123, corresponding to SEQ ID NO:3), human β-arrestin 2 (GenBank™ Accession No. AAC99468, corresponding to SEQ ID NO: 4), human arrestin C (GenBank™ Accession No. AAC78395, corresponding to SEQ ID NO: 5), and human X arrestin (GenBank™ Accession No. AAB84302, corresponding to SEQ ID NO: 6), said mutant having either a mutation within the phosphorylation recognition domain or having a truncation of all or a part of the C-terminal regulatory domain.

As used herein, an “isolated” or “purified” arrestin mutant, or biologically active portion thereof, is substantially free of cellular material or other contaminating proteins from the cell or tissue source from which the mutant is derived, or substantially free from chemical precursors or other chemicals when chemically synthesized. The language “substantially free of cellular material” includes preparations of arrestin mutant in which the protein is separated from cellular components of the cells from which it is isolated or recombinantly produced. In one embodiment, the language “substantially free of cellular material” includes preparations of arrestin mutant having less than about 30% (by dry weight) of non-arrestin mutant protein (also referred to herein as a “contaminating protein”), more preferably less than about 20% of non-arrestin mutant protein, still more preferably less than about 10% of non-arrestin mutant protein, and most preferably less than about 5% non-arrestin mutant protein. When the arrestin mutant or biologically active portion thereof is recombinantly produced, it is also preferably substantially free of culture medium, i.e., culture medium represents less than about 20%, more preferably less than about 10%, and most preferably less than about 5% of the volume of the mutant preparation.

The language “substantially free of chemical precursors or other chemicals” includes preparations of arrestin mutant protein in which the protein is separated from chemical precursors or other chemicals which are involved in the synthesis of the protein. In one embodiment, the language “substantially free of chemical precursors or other chemicals” includes preparations of arrestin mutant protein having less than about 30% (by dry weight) of chemical precursors, more preferably less than about 20% chemical precursors, still more preferably less than about 10% chemical precursors, and most preferably less than about 5% chemical precursors.

As used herein, a “biologically active portion” of an arrestin mutant protein includes a fragment of an arrestin mutant which participates in an interaction between the mutant protein molecule and, for example, a GPCR. Biologically active portions of arrestin mutants protein include peptides comprising amino acid sequences sufficiently identical to or derived from the amino acid sequence of the arrestin mutant, e.g., the amino acid sequence shown in SEQ ID NO: 7 of SEQ ID NO: 8, which include less amino acids than the full length arrestin mutant proteins, and exhibit at least one activity of an arrestin mutant protein. Biologically active portions of an arrestin mutant protein can be substituted for full-length arrestin proteins in the screening assays of the present invention, e.g., in screening assays for agents which bind GPCRs.

Arrestin mutant proteins of the present invention can be made according to recombinant techniques. In general, recombinant production of arrestin mutant protein can be accomplished by inserting the coding sequence of the gene encoding a arrestin mutant protein into a recombinant expression vector in the context of the appropriate regulatory elements, such that expression of the recombinant arrestin mutant protein is achieved. The arrestin mutant proteins further can be purified from cells. Such cells include, but are not limited to, bacterial cells, yeast cells, fungal cells and mammalian cells. Such a procedure is described in detail, for example, in Example 1. Purification of arrestin mutant proteins from cells can be accomplished according to the procedures described herein for the isolation of arrestin mutant proteins or, alternatively, can be purified from appropriate biological sources.

Isolated nucleic acid molecules which encode the phosphorylation-independent mutants of the present invention are also within the scope of the invention. The term “isolated nucleic acid molecule” includes nucleic acid molecules which are substantially free of other cellular material when purified, for example, from biological samples (e.g., tissue samples), or substantially free of culture medium when produced by recombinant techniques, or substantially free of chemical precursors or other chemicals when chemically synthesized. Moreover, an “isolated” nucleic acid molecule, such as a cDNA molecule, can be one which is separated from other nucleic acid molecules which are present in the natural source of the nucleic acid. For example, an “isolated” nucleic acid molecule, such as a cDNA molecule, can be one which is separated from the chromosome with which the corresponding genomic DNA is naturally associated. Preferably, such an “isolated” nucleic acid is free of sequences which naturally flank the nucleic acid (i e., sequences located at the 5′ and 3′ ends of the nucleic acid) in the genomic DNA of the organism from which the nucleic acid is derived.

Also included within the scope of the invention are such phosphorylation-independent muatnt-encoding nucleic acid molecules includes within a vector (e.g., included within an expression vector) as well as such vectors included within a host cell.

B. Preparation of GPCR Proteins

GPCR proteins of the present invention can also be produced using recombinant protein expression technologies. For example, the GPCRs of the present invention can be produce in organisms including bacteria (e.g., Escherichia coli) yeast (e.g., Saccharomyces cerevisiae or Candida parapsilosis) and/or mammalian cells. Production of GPCRs in E. coli can been accomplished with slight modification of standard recombinant expression procedures e.g., those used to recombinantly produce globular proteins) taking into account the fact that GPCRs are integral membrane proteins. For example, the anionic detergent, sarkosvl, can be utilized to enable the extraction of the expressed GPCRs from inclusion bodies (when GPCRs are produced in E. coli) in a form that can be functionally reconstituted into membrane preparations.

C. Alembrane Preparations

Membrane preparations, for example, receptor-containing preparations (e.g., preparations containing GPCRs), can be prepared according to any art recognized methodologies of membrane preparation (e.g., according to Burgisser et al. (1985) Biochem. Biophys. Res. Commun. 133:1202-1209). As used herein, the term “membrane preparation” includes. In general, processes for preparing membrane preparations involves the obtaining of suitable cellular or tissue material (e.g., eukaryotic tissues or cell cultures, for example, cultures cells expressing or overexpressing GPCRs, in particular, orphan GPCRs). This is followed by cellular disintegration, for example, by homogenization, ultrasonics, detergent or organic solvent lysis, agitation, high temperature or some other suitable procedure, followed by separation of cytoplasmatic components (e.g., soluble components) and/or separation of coarser particles (e.g., by centrifuging, filtering, and the like). In an exemplary embodiment, membrane preparations of the present invention can be prepared according to the methodologies described in “Reconstitution of receptor-promoted signal transduction using purified receptor, G protein, and effector” Berstein and Biddlecome (2000) in “G protein coupled receptors”, eds. Haga and Berstein, pp. 117-154, CRC Press LLC, Boca Raton, Fla.

Alternatively, membrane preparations for use in the assays of the present invention can be reconstituted from, for example, membranes prepared according to any of the above-described technologies and purified or isolated GPCRs. A general GPCR purification scheme can involve solubilization of GPRCs with a non-ionic detergent, chromatography on hydroxylapatite, followed in some cases, by SH-affinity chromatography. (See, for example, Wohlrab (1986) Biochim. Biophys. Acta 853:8170-8173; Wehrle and Pedersen (1989) J. Membr. Biol. 111:199-213; Kramer. and Palmeri (1989) Biochim. Biophys. Acta 974:1-23; Kolbe et al. (1 984) J. Biol. Chem. 259:9115-9120; Kaplan et al. (1986) J. Biol. Chem. 261:12767-12773; andGuerinetal. (1990) J Biol. Chem. 265: 19736-19741). An alternative GPCR purification scheme involves solubilization of GPCRs with a non-ionic detergent, chromatography on hydroxylapatite, followed by sequential chromatography on Matrex Gel Orange A™, Matrix Gel Blue B™, and Affi-Gel 501™. (See, for example, Kaplan et al. (1990) J. Biol. Chem. 265:13379-13385.)

Examples of such non-ionic detergent solubilizing agents include digitonin, n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether), ₙ3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

An exemplary protocol for preparing membranes from cells expressing recombinant GPCRs is as follows: Solutions: a. Solution A: 20 mM Tris-Cl (pH 8), 1 mM EDTA, 1 mM EGTA, and PI (protease inhibitors: 0.1 phenylmethylsulfonylfluoride, 2 microg/ml aprotinin, and 10 microg/ml leupeptin); b. Solution B: 20 mM Tris-Cl (pH 8), 3 mM MgCl2, 10 microg/ml deoxyribonuclease I, and PI. 1. Harvest cells by centrifugation at 1000×g for 10 min. Discard supernatant. 2. Resuspend cell pellet with ice-cold solution A. 3. Homogeneize the cell suspension with a Dounce homogeneizer, 10 strokes. 4. Centrifuge homogenate at 30,000×g for 20 min. Discard supernatant. 5. Repeat steps 3. and 4. with the pellet. 6. Resuspend pellet (membrane fraction) with a small volume of solution B with a Dounce homogeneizer. 7. Aliquot as needed and store at −80 degree C. until use.

D. Labeled Reactants

Exemplary labels for labeling reactants for use in the assay methods of the present invention include radiolabels (e.g., radioactive isotopes including but not limited to ³⁵S, ¹⁴C, ³H, ³²P and ¹²⁵I), colorometric labels (e.g., chromophores), reporter proteins and fluorescent labels (e.g., fluorophores). Such labels as well as methods of incorporating such labels are known in the art.

E. Test Compounds

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA. 90:6909; Erb et al. (1994) Proc. Natl. Acad. Sci. USA 91:11422; Zuckermnann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g.? Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl Acad. Sci. 87:6378-6382); (Felici (1991) J. Mol. Biol. 222:301-310); (Ladner supra.).

Exemplary compounds which can be screened for activity include, but are not limited to, peptides, nucleic acids, carbohydrates, small organic molecules, and peptidomimetics. The term “peptidomimetic” is intended to encompass compounds that are comprised, at least in part, of molecular structures different from naturally-occurring L-amino acid residues linked by natural peptide bonds. “Peptidomimetics” are intended to include compounds composed, in whole or in part, of structures such as D-amino acids, non-naturally-occurring L-amino acids, modified peptide backbones and the like, as well as compounds that are composed, in whole or in part, of molecular structures unrelated to naturally-occurring L-amino acid residues linked by natural peptide bonds.

F. Automated Screening Methods

The methods of the present invention may be automated to provide convenient, real time, high volume methods of screening test compounds for the ability to bind to or modulate GPCRs (e.g., screening for surrogate ligands of orphan GPCRs or modulators of GPCRs). Automated methods can be optimized to detect binding of labeled assay components (e.g., labeled arrestin mutant protein) to GPCR, optionally in the presence or absence of known GPCR agonist, or to detect, for example, binding of unlabeled assay components, optionally in the presence or absence of known GPCR agonist. The binding of assay components can be detected over time (i.e., comparing an assay reaction mixture before and after contacting with a test compound or plurality of test compounds), or by comparison of a test compound or mixture of test compounds to a control assay reaction mixture which is not exposed to the test compound(s), or by comparison to pre-established indicia. Both qualitative assessments (positive/negative) and quantitative assessments (comparative levels of binding) may be provided by the present automated methods, as will be apparent to those skilled in the art. The present invention thus further features methods and/or apparatus for automated screening of GPCR ligands (e.g., surrogate ligand identification) or modulators, by detecting the binding of arrestin mutant protein to GPCR in response to test compound(s), optionally in the presence or absence of GPCR agonist. Arrestin binding may be indicated by an alteration in the distribution of a detectable signal within an assay reaction mixture over time, between a test reaction mixture and a control reaction mixture, or by comparison to previously established parameters. Measurement points may be over time, or among test and control samples or mixtures.

A computer program may be used to process samples, record output, and/or process data obtained by carrying out the above-described steps. The preferred computer program product comprises a computer readable storage medium having computer-readable program code means embodied in the medium. Hardware suitable for use in such automated apparatus will be apparent to those of skill in the art, and may include computer controllers, automated sample handlers, surface plasmon resonance (SPR) measurement tools (e.g., BIACORE™ instruments), tools for measuring bound and/or free labeled reactants, printers and optical displays. The hardware may also contain a computer-controlled stepper motor so that each control and/or test sample can be arranged as an array of samples and automatically and repeatedly positioned for detection of binding of reagents. The measurement tool is preferably operatively coupled to a general purpose or application specific computer controller. The hardware may also comprise a computer program product for controlling operation of the measurement tool and performing numerical operations relating to the above-described steps. For example, a controller may accept set-up and other related data via a file, disk input or data bus. A display and printer may also be provided to visually display the operations performed by the controller. It will be understood by those having skill in the art that the functions performed by the controller may be realized in whole or in part as software modules running on a general purpose computer system. Alternatively, a dedicated stand-alone system with application specific integrated circuits for performing the above described functions and operations may be provided. As provided above, detection of binding of assay reagents may take the form detection of binding of labeled reactants or unlabeled reactants, although those skilled in the art will appreciate that other indicia are known and may be used in the practice of the present invention, such as may be provided by labels that produce signals detectable by, for example, X-ray diffraction or absorption or magnetism. Such labels include, for example, electron-dense reagents.

G. Cell-Based Assays and Labeled Reactants

Another aspect of the invention features cell-based assays for identifying reagents (e.g., ligands, surrogate ligands, agonists, antagonists and the like) which bind to and/or modulate the activity of GPCRs. In one embodiment, the invention features a method of identifying compounds which bind to and/or modulate the activity of a GPCR that involves contacting a cell which expresses a GPCR and an arrestin mutant of the present invention (e.g., a mutant that interacts with the GPCR in a phosphorylation independent manner) with a test compound and determining the effect of the compound on the interaction between the GPCR and the arrestin mutant, such that a reagent that binds to and/or modulates the activity of the GPCR is identified. Preferably, the interaction between GPCR and arrestin mutant is detectable by labeling one or both of the GPCR and arrestin mutant. In an exemplary embodiment, the interaction between GPCR and arrestin mutant is detectable by labeling the GPCR and/or arrestin mutant with labels detectable as a result of their interaction (e.g., label:label interaction) preferably reagents designed for detecting protein:protein interactions in live cells. For example, bioluminescence resonance energy transfer (BRET) can be used to determine the interaction between GPCR and arrestin mutant. In a preferred embodiment, the arrestin mutant (or a biologically active portion thereof) is recombinantly fused to a BRET donor (e.g., Renilla luciferase) or acceptor (e.g., green fluorescent protein) and energy transfer between labeled arrestin mutant and labeled GPCR (e.g., labeled with acceptor or donor, respectively) is detected. Such a technology is described, for example, in Angers et al. (2000) Proc. Natl. Acad. Sci. 97:3684-3689, the content of which is incorporated by this reference. In another exemplary embodiment, the GPCR and/or arrestin mutant can be labeled with inactive, weakly-complementing β-galactosidase mutants according to Intercistronic Complementation Analysis Screening Technology (“ICAST”). Association between the molecules of interest (e.g., GPCR and arrestin mutant) brings the complementing β-galactosidase mutants into proximity so that complementation occurs and active β-galactosidase is produced. The active β-galactosidase may be detected by methods well-known in the art. Such a technology is described in, for example, WO 98/44350, the content of which is incorporated by this reference. The ICAST technology is also suitable for detection of arrestin mutant:GPCR interaction in vitro (e.g., in the cell-free assays described herein).

Preferably, in the cell-based assays (as well as in vitro assays) described herein, determining the effectiveness of a test compound is performed in the presence or absence of the test compound, in the presence of the test compound as compared to a suitable control (e.g., a buffer or solvent control, known positive or negative control compounds) or in the presence of the test compound as compared to a predetermined or known range or value for binding and/or activity. Moreover, cell-based agonist assays are preferably performed in the presence of a known ligand specific for the GPCR of interest or, optionally, a surrogate ligand for the GPCR of interest.

### IV Preparation and Administration of Pharmaceutical Compositions of GPCR Ligands and/or Modulators

A. Pharmaceutical Preparations of Identified Compounds

After identifying certain test compounds as potential modulators of GPCR activity, the practitioner of the subject assay will continue to test the efficacy and specificity of the selected compounds both in vitro and in vivo as described further herein. Whether for subsequent in vivo testing, or for administration to an animal as an approved drug, compounds identified in the subject assay, or a pharmaceutically acceptable salt thereof, can be formulated in pharmaceutical preparations for in vivo administration to an animal, preferably a human. Such preparations comprise a therapeutically (or prophylactically) effective amount of the GPCR modulator or ligand, and a pharmaceutically acceptable carrier or excipient. The preparations may be formulated for administration with a pharmaceutically acceptable carrier except insofar as any conventional media or agent is incompatible with the activity of the compound. As used herein, “pharmaceutically acceptable carrier” includes any and all solvents or wetting agents, emulsifying or dispersion media, and the like which may be appropriate for the desired route of administration of the pharmaceutical preparation. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Other carriers include water, saline, saline buffered with phosphate or citrate at suitable concentrations so as to obtain at all times isotonic pharmaceutical preparations of the desired pH, (for example, neutral pH), dextrose, ethanol, mannitol, polyol (for example, glycerol, propylene glycol, liquid polyethylene glycol and the like) or suitable mixtures thereof. The use of other such media for pharmaceutically active substances is known in the art. The optimum concentration of the active ingredient(s) in the chosen medium can be determined empirically, according to procedures well known to medicinal chemists. The carrier and composition can be sterile. The formulation should suit the mode of administration.

B. Effective Doses of Identified Compounds for Administration

Single and multiple (e.g., 5 to 7 days) dose investigative toxicology studies are typically performed in the efficacy test species using the intended route of administration for the efficacy study. These investigative toxicology studies are performed to identify maximum tolerated dose, subjective bioavailability from the intraperitoneal or oral routes of administration, and estimation of an initial safety margin. Initial bioavailability and pharmacokinetics (blood clearance) of the compounds may be determined, with standard cold or radioactive assay methods, to assist in defining appropriate dosing regimens for the compounds in the animal models.

The effective dose of the GPCR modulator or ligand will typically be in the range of about 0.01 to about 50 mg/kgs, preferably about 0.1 to about 10 m g/kg of mammalian body weight, administered in single or multiple doses. Generally, the GPCR modulator or ligand may be administered to patients in need of such treatment in a daily dose range of about 0.5 to about 2000 mg per patient.

The amount of GPCR modulator or ligand which will be effective in the treatment or prevention of a particular disorder or condition will depend on the nature of the disorder or condition, and can be determined by standard clinical techniques. In addition, in vitro or in vivo assays may optionally be employed to help identify optimal dosage ranges. Effective doses may be extrapolated from dose-response curves derived from in vitro or animal model test systems. The precise dosage level of GPCR modulator or ligand, as the active component(s), should be determined by the attending physician or other health care provider and will depend upon well known factors, including route of administration, and the age, body weight, sex and general health of the individual; the nature, severity and clinical stage of the disease; and the use (or not) of concomitant therapies.

Patients in need of treatment with a GPCR modulator include patients having Retinitis pigmentosa, stationary night blindness, color blindness, nephrogenic DI, isolated glucocorticoid deficienct, hyperfunctioning thyroid adenomas, familial hypocalciuric hypercalcemia, hyperparathyroidism, nerulogical disorders and the like.

C. Administration of Identified Compounds

Various delivery vehicles are known and can be used to administer the GPCR modulator or ligand, e.g., encapsulation in liposomes, microparticles, injectable “deposit formulations”, microcapsules, etc. Materials and methods for producing the various formulations are well known in the art (see e.g. U.S. Pat. Nos. 5,182,293 and 4,837,311 (tablets, capsules and other oral formulations as well as intravenous formulations)). Suitable vehicles and their formulation inclusive of other proteins are described, for example, in the book Remington's Pharmaceutical Sciences (Remington's Pharmaceutical Sciences. Mack Publishing Company, Easton, Pa., USA 1985). Administration can be systemic or local. Methods of introduction include but are not limited to pulmonary administration, intradermal, intramuscular, intraperitoneal, intravenous, subcutaneous, intranasal, epidural and oral routes, for example by infusion or bolus injection, by absorption through epithelial or mucocutaneous linings (e.g., oral mucosa, rectal and intestinal mucosa, etc.) and may be administered together with other biologically active agents. Typically, compositions for intravenous administration are solutions in sterile isotonic aqueous buffer. Where necessary, the composition may also include a solubilizing agent and a local anesthetic to ease pain at the side of the injection. Generally, the ingredients are supplied either separately or mixed together in unit dosage form, for example, as a lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. Topical administration to an individual of an effective amount of the biased agonist can be accomplished by administering the compound(s) directly to the affected area of the skin of the individual. For this purpose, the GPCR modulator or ligand is administered or applied in a composition including a pharmacologically acceptable topical carrier, such as a gel, an ointment, a lotion, or a cream, which includes, without limitation, such carriers as water, glycerol, alcohol, propylene glycol, fatty alcohols, triglycerides, fatty acid esters, or mineral oils.

Other topical carriers include liquid petroleum, isopropyl palmitate, polyethylene glycol, ethanol (95%), polyoxyethylene monolaurate (5%) in water, or sodium lauryl sulfate (5%) in water. Other materials such as anti-oxidants, humectants, viscosity stabilizers, and similar agents may be added as necessary.

In cases where it is desirable to administer the GPCR modulator or ligand locally to the area in need of treatment this may be achieved by, for example, and not by way of limitation, local infusion during surgery, topical application, by injection, by means of a catheter, by means of a suppository, or by means of a skin patch or implant, said implant being of a porous, non-porous, or gelatinous material, including membranes, such as sialastic membranes, or fibers which release the compound into the skin, by either passive or active release mechanisms.

This invention is further illustrated by the following examples which should not be construed as limiting. The contents of all patents, published patent applications and references cited throughout this application, including Kovoor et al. (1999) J. Biol. Chem. 274:6831-6834, are incorporated herein by reference. The Figures and the Sequence Listing provided herewith are also incorporated herein by reference.

### EXAMPLES

### Examples 1

### Generation and Biological Characterization of Phosphorylation-Independent Arrestin Mutants

Example 1 describes the generation of various phosphorylation-independent mutants and characterization of the biological activity of said mutants in vitro. The following materials and methods were used in carrying out the experiments in Example 1 and are further applicable to the methodologies described, for example, in Examples 2-3 described below, as well as in the identification of additional phosphorylation-independent muatnts.

Mutagenesis and Biochemical Characterization of β-Arrestins—Mutations Arg169→Glu (CGG→GAG), Gln394→Ter (CAA→TAA), and Asp383→Ter (GAT→TAG) were introduced by polymerase chain reaction in β-arrestin construct pBARR (Gurevich et al. (1995) J. Biol. Chem. 270:720-731), that was used for in vitro transcription and translation, as described (Gurevich et al. (1995), supra). NcoI/HindIII 1404-base pair open reading frame was then subdloned into appropriately digested Escherichia coliexpression vector pTrcHisB (Invitrogen). All β-arrestin species were expressed in the in vitro translation system and tested in the direct binding assay (Gurevich et al. (1995), supra), overexpressed in E. coli, purified to apparent homogeneity (Gray-Keller et al. (1997) Biochemistry 36:7058-7063), and characterized in the agonist affinity shift assay (Gurevich et al. (1997) J. Biol Chem. 272:28849-28852), essentially as described.

Direct Binding Assay—In vitro translated tritiated arrestins (50 fmol) were incubated in 50 mM Tris-HCl, pH 7.5, 0.5 mM MgCl2, 1.5 mM dithiothreitol, 50 mM potassium acetate with 7.5 pmol of the various functional forms of rhodopsin or with P-β₂AR or β₂AR (100 fmol/assay) in a final volume of 50 μl for 5 min at 37° C. in room light (rhodopsin) or for 60 min at 30° C. in the presence of 0.1 mM -agonist isoproterenol. The samples were immediately cooled on ice and loaded onto 2 ml Sepharose 2B columns equilibrated with 20 mM Tris-HCl, pH 7.5, 2 mM EDTA. Bound arrestin eluted with receptor-containing membranes in the void volume (between 0.5 and 1.1 ml). Nonspecific binding determined in the presence of 0.3 μg of liposomes was subtracted.

Agonist Affinity ShiftAssay—P-β₂AR or β₂AR (10-15 fmol/assay) was incubated in 0.25 ml of 10 mM Tris-HCl, pH 7.4, 100 mM NaCl (buffer A) containing 0.1 mg/ml bovine serum albumin in the presence of 65-75 fmol of [¹²⁵I] iodopindolol (NEN Life Science Products) and the indicated concentrations of arrestins and agonists for 60 min at 22° C. Samples were then cooled on ice and loaded at 4° C. onto 2 ml of Sephadex G-50 columns. Receptor-containing liposomes with bound radioligand were eluted with buffer A (between 0.6 and 1.5 ml), and radioactivity was quantitated in a liquid scintillation counter. Nonspecific binding was determined in the presence of 10 μM alprenolol. All experiments were repeated two to three times, and data are presented as means ±S.D.

It is now understood that arrestin proteins play a key role in the desensitization of GPCRs. It also is now appreciated that naturally-occurring arrestins exhibit selectivity for the activated phosphorylated forms of GPCRs (see e.g., Gurevich and Benovic (1993) J. Biol. Chem. 268:11628-11638; Gurevich et al. (1995) J. Biol. Chem. 270:720-731; and Gurevich et al. (1994) J. Biol. Chem. 270:720-731). In particular, results of in vitro studies indicate that the arrestins have two primary binding sites for GPCRs: an activation- recognition site or region that recognizes the agonist-activated state of the receptor and a phosphorylation-recognition site or region that interacts with phosphorylated elements (e.g., GRK-phosphorylated elements) of the receptor. A potent secondary receptor-binding site is mobilized for the interaction only when both primary sites are simultaneously engaged, i.e. when an arrestin encounters activated and phosphorylated receptor. According to this model of arrestin:GPCR interaction, arrestin is kept in its basal conformation by several intramolecular interactions in which certain residues in the primary binding sites (“trigger” residues) are involved. One of these triggers is pulled by binding of the arrestin to an activated form of the receptor, the other, by the interaction of arrestin with phosphate(s) introduced by GRK, for example.

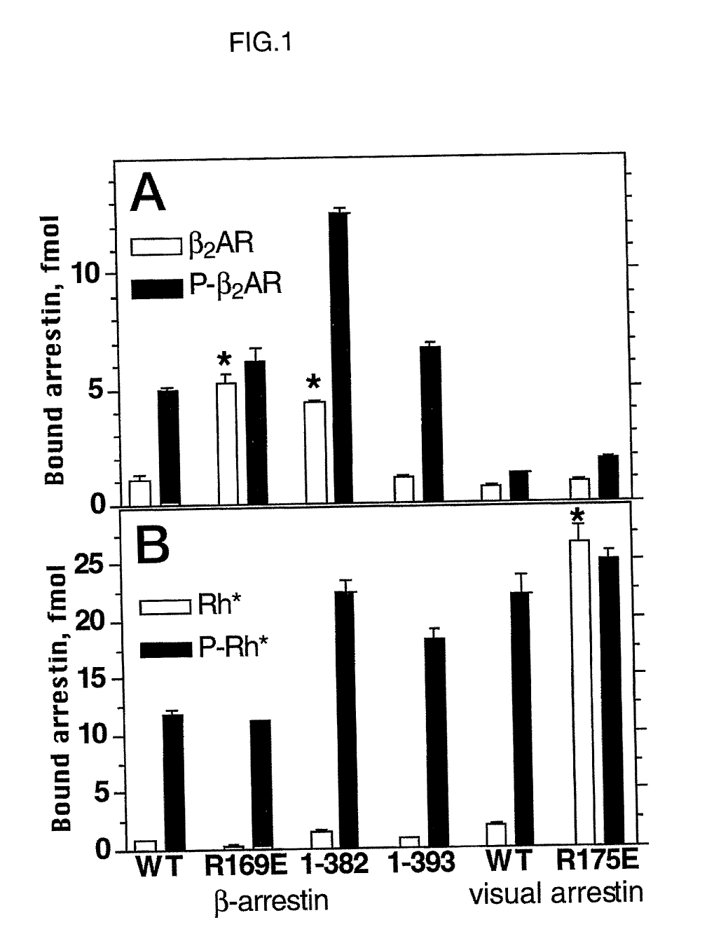

In order to confirm the validity of the model, three bovine β-arrestin mutants were constructed: 1) Arg169→Glu, that reverses the charge of the putative phosphorylation-sensitive trigger (a similar mutation in visual arrestin is described, for example, is Gurevich and Benovic (1995) J. Biol. Chem. 270:6010-6016); 2) Gln394→Ter; and 3) Asp383→Ter, that delete a part or all of the regulatory arrestin COOH terminus, which keeps arrestin in a basal conformation and suppresses an untimely mobilization of the secondary binding site (Gurevich (1998) J. Biol. Chem. 27315501-15506). First, the ability of these mutants to interact with purified β₂AR reconstituted into phospholipid vesicles was tested by performing direct binding studies (Gurevich et al. (1995) supra) and agonist affinity shift assays (Gurevich et al. (1997) supra) in vitro. Wild type β-arrestin and β-arrestin-(1-393) bind poorly to activated unphosphorylated receptor (FIG. 1A). In contrast, β-arrestin-(Arg169-Glu) and β-arrestin-(1-382) demonstrate significantly higher binding to activated unphosphorylated receptor (FIG. 1A). Wild type β-arrestin and all three mutants readily bind to activated and phosphorylated β₂AR (FIG. 1 A). Thus, β-arrestin-(Arg169→Glu) and β-arrestin-(1-382) bind to activated β₂AR in a phosphorylation-independent fashion.

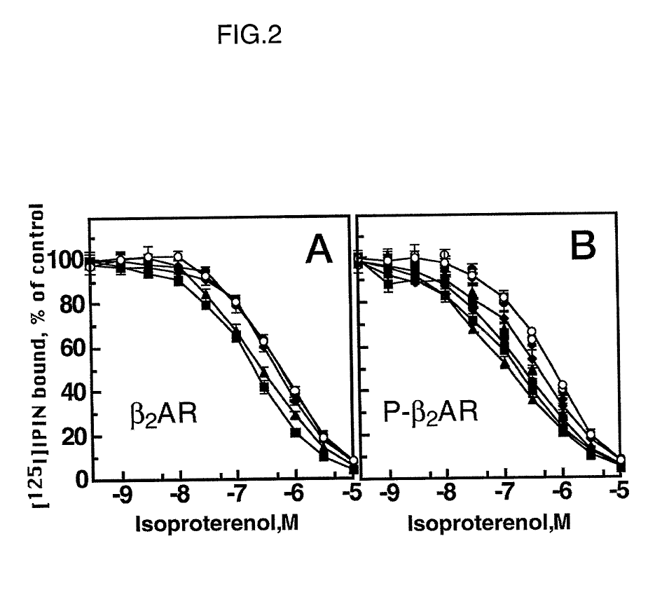

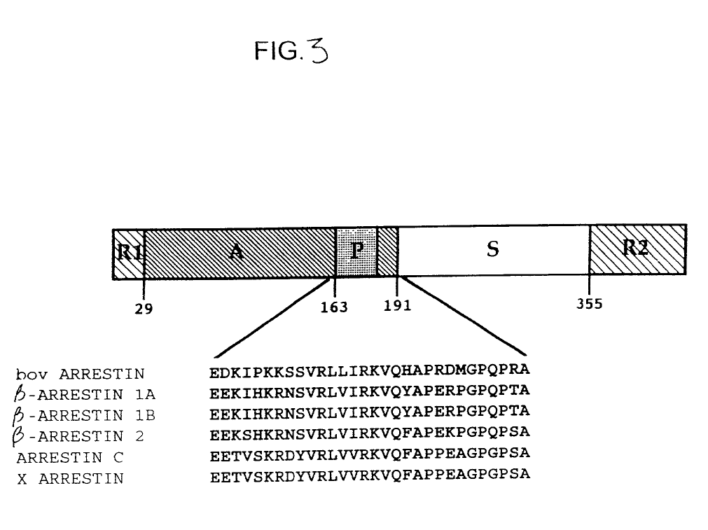

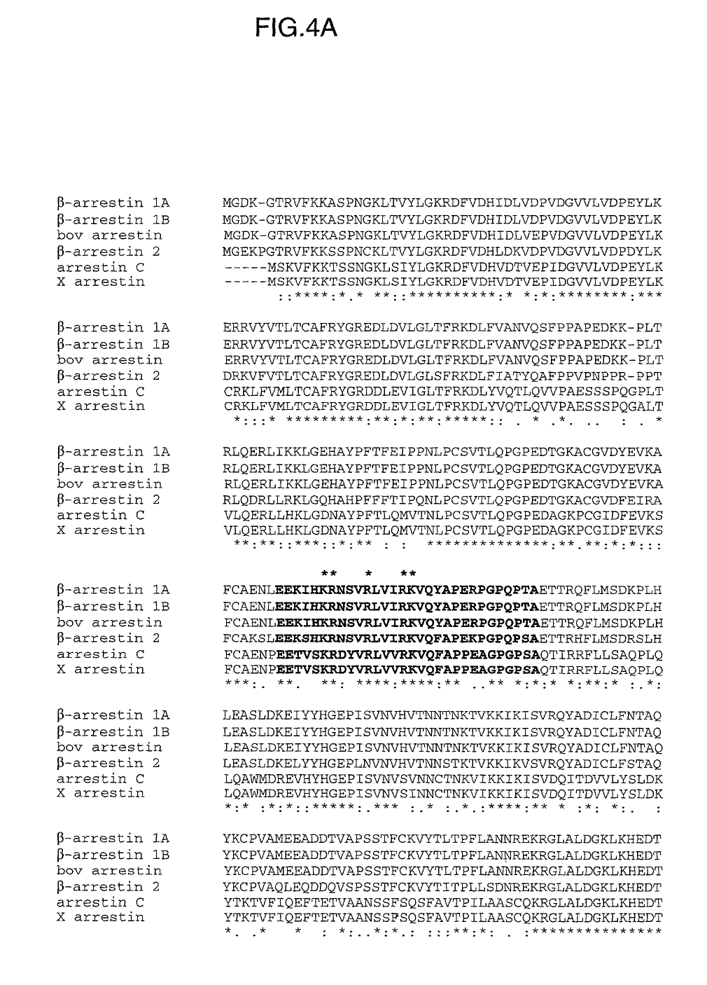

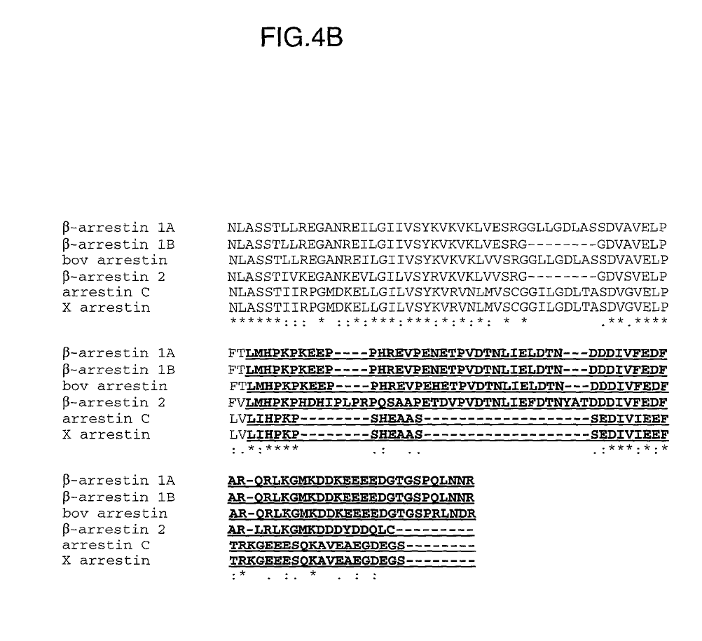

It was recently reported that arrestin-receptor complex is similar to G protein-receptor complex in two respects: agonists have higher affinity for arrestin-receptor complex than for receptor alone, and only a fraction of the receptors forms such a high agonist affinity complex (HAC) even at saturating concentrations of arrestin (Gurevich et al. (1997) supra). The maximum percentage of the receptor in HAC gives a good estimate of the propensity of a given arrestin protein to bind tightly to the receptor (arrestin competency) Gurevich et al. (1997) supra)). Consistent with the direct binding data (FIG. 1A), β-arrestin-(Arg169→Glu) and β-arrestin-(1-382) induced the formation of HAC by unphosphorylated β₂AR (22±4% in both cases) (FIG. 2A). In contrast, all forms of β-arrestin induced the formation of HAC by phosphorylated β₂AR (P-β₂AR) (FIG. 2B). The percentage of HAC formed by P-β₂AR in the presence of saturating (1 μM) concentration of β-arrestin, β-arrestin-(Arg169→Glu), β-arrestin-(1-382), and β-arrestin-(1-393) was 31±6, 52±3, 41±3, and 20±4%, respectively (FIG. 2B). In summary, in both in vitro assays β-arrestin-(Arg169→Glu) and β-arrestin-(1-382) demonstrate constitutive activity (phosphorylation-independent receptor binding).

### Examples 2-3

### Screening Assays Utilizing GPCRs and Phosphorylation-Independent Arrestin Mutants

The present invention is based at least in part, on the discovery that that phosphorylation-independent arrestin mutants are capable of bind GPCRs in a manner independent of any requirement for phosphorylation. It is now understood that this feature of the phosphorylation-independent mutants of the present invention that makes them particularly suited for use in in vitro screening assays. Accordingly, the present invention features screening assays designed to take advantage of the fact that phosphorylation-independent arrestin mutants can bind receptor (e.g., GPCRs) in a manner independent of cellular receptor kinases. Examples 2-3 describe assays that are directed to identify natural and surrogate agonists for orphan GPCRs using phosphorylation-independent mutant human β-arrestins.

### Example 4

### High Throughput Screening Assays (HTS Assays)

Radiolabelled in vitro translated mutant human β-arrestin 2 Arg170→Glu and mutant human β-arrestin (1-382) are prepared as follows: A mutation at the codon encoding Arg170 is introduced in the cDNA sequence of human β-arrestin 2 by polymerase chain reaction in a suitable β-arrestin construct resulting in a phosphorylation recognition domain mutant. In a similar fashion, a mutation at the codon encoding amino acid 382 of human β-arrestin 2 is introduced by polymerase chain reaction in a suitable β-arrestin construct encoding a stop codon (→TAG) resulting in Asp383→Ter. The mutant constructs are then used for in vitro transcription and translation, as described herein. In vitro translated Arg 170→Glu or Asp383→Ter is then radiolabelled to a suitable specific activity. High specific activity (>500 Ci/mmol) labelled mutants are preferred for subsequent use in screening assays.

A membrane preparation is prepared from cells that overexpress an orphan GPCR. Suitable cells for overexpression of orphan GPCRs include Sf9 cells and/or other mammalian cells suitable for overexpression of GPCRs. An aliquot of membrane preparation is contacted with an aliquot of radiolabelled in vitro translated Arg 170→Glu or Asp383→Ter β-arrestin mutant and the resulting mixture is contacted with a an aliquot of test compounds derived from a small molecule library. Specific binding is detected following a period of incubation of reactants by washing unbound radiolabelled in vitro translated Arg 170→Glu or Asp383→Ter β-arrestin mutant from the well. Assay conditions (e.g., concentration of assay components, composition of assay buffer, temperature and time of incubation) are optimized such that a signal-to-noise ratio of >10-fold is achieved. Washing procedures such as high-salt or urea treatment may be used to achieve the desired signal-to-noise ratios.

Equal aliquots of membrane prep containing the orphan GPCR are distributed in the wells of a multiwell reaction vessel as are equal aliquots of radiolabelled in vitro translated Arg 170→Glu or Asp383→Ter β-arrestin mutant. Multiple wells are then reacted with different aliquots of test compounds. each aliquot containing a distinct mixture of potential surrogate ligands.

### Example 5

### BIACORE Screening Assays

Unlabelled in vitro translated mutant human β-arrestin 2 Arg 170→Glu and mutant human β-arrestin (1-382) are prepared as described in Example 4.

A membrane preparation is prepared from cells that overexpress an orphan GPCR. An aliquot of membrane preparation is immobilized on a sensor chip and the immobilized membrane prep is contacted with an aliquot of in vitro translated Arg 170→Glu or Asp383→Ter β-arrestin mutant. The resulting mixture is contacted with a an aliquot of test compounds derived from a small molecule library. Specific binding is detected following a period of incubation of reactants by detecting a change in surface plasmon resonance (SPR) signals generated from the reaction before and after the addition of test compound. Assay conditions are optimized such that a signal-to-noise ratio of >3-fold is achieved.

Equal aliquots of membrane prep containing the orphan GPCR are distributed on sensor chips and contacted with equal aliquots of in vitro translated Arg 170→Glu or Asp383→Ter β-arrestin mutant. Sensor chips are then reacted with different aliquots of test compounds, each aliquot containing a distinct mixture of potential surrogate ligands.

### Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method of identifying a G protein-coupled receptor (GPCR) ligand comprising contacting a composition comprising the GPCR and a constitutively active arrestin mutant with a test compound and determining the ability of the test compound to modulate binding of the arrestin mutant to the GPCR, wherein modulation of binding indicates that the test compound is a GPCR ligand.

2. The method of claim 1, wherein the GPCR is an orphan receptor.

3. The method of claim 2, wherein the ligand identified is a surrogate ligand.

4. A method of identifying a G protein-coupled receptor (GPCR) antagonist comprising contacting a composition comprising the GPCR, a phosphorylation-independent arrestin mutant and a GPCR agonist with a test compound and determining the ability of the test compound to modulate binding of the arrestin mutant to said GPCR, wherein modulation of binding indicates that the test compound is a GPCR antagonist.

5. The method of claim 1 or 4, wherein the constitutively active arrestin mutant is a phosphorylation-independent mutant.

6. The method of claim 1 or 4, wherein the constitutively active arrestin mutant has a mutation if the phosphorylation recognition domain.

7. The method of claim 1 or 4, wherein the constitutively active arrestin mutant has a truncation of the C-terminal regulatory domain.

8. The method of claim 6, wherein the constitutively active arrestin mutant has a mutation in an amino acid corresponding to any one of Arg171, Arg175, Lys 167 and Lys176 of SEQ ID NO: 4.

9. The method of claim 6, wherein the constitutively active arrestin mutant has a mutation in an amino acid corresponding to Arg175 of SEQ ID NO: 4.

10. The method of claim 6, wherein the constitutively active arrestin mutant has the amino acid sequence of SEQ ID NO: 7.

11. The method of claim 7, wherein the constitutively active arrestin mutant has the amino acid sequence of SEQ ID NO: 8.

12. The method of claim 1 or 4, wherein the composition is a membrane preparation.

13. The method of claim 4, wherein the GPCR is a β-adrenergic receptor.

14. The method of claim 4, wherein the GPCR is a visual receptor.

15. The method of claim 1 or 4, wherein the arrestin mutant is a visual arrestin mutant.

16. The method of claim 1 or 4, wherein the arrestin mutant is a β arrestin mutant.

17. The method of claim 1 or 4, wherein the arrestin mutant is an arrestin C mutant.

18. The method of claim 1 or 4, wherein the composition further comprises a second GPCR.

19. The method of claim 1 or 4, wherein the composition further comprises a plurality of other GPCRs which differ from said GPCR.

20. The method of claim 1 or 4, wherein the test compound is in an aqueous solution.

21. The method of claim 1 or 4, wherein the test compound is in an organic solution.

22. The method of claim 1 or 4, wherein the test compound is in a mixture of a plurality of test compounds.

23. The method of claim 1 or 4, wherein said arrestin mutant is conjugated to a detectable marker.

24. The method of claim 1 or 4, wherein the detectable marker is a radioisotopic label.

25. The method of claim 1 or 4, wherein the detectable marker is a colorometric label.

26. The method of claim 1 or 4, wherein the detectable marker is a reporter protein.

27. The method of claim 1 or 4, wherein determining the ability of the test compound to modulate binding of the arrestin mutant to the GPCR comprises detecting a change in surface plasmon resonance

28. The method of claim 22, wherein the mixture is a small molecule library.

29. A method of identifying a G protein-coupled receptor (GPCR) ligand comprising contacting a GPCR with a phosphorylation-independent arrestin mutant and a test compound and determining the ability of the test compound to modulate binding of the arrestin mutant to the GPCR, wherein modulation of binding indicates that the test compound is a GPCR ligand.

30. A method of identifying a surrogate ligand for an orphan G protein-coupled receptor (GPCR) comprising contacting a membrane preparation comprising said orphan GPCR and a phosphorylation-independent arrestin mutant with a test compound and determining the ability of the test compound to effect binding of said arrestin mutant to said orphan GPCR.

31. A phosphorylation-independent β arrestin mutant.

32. The mutant of claim 31, having a mutation in the phosphorylation recognition region.

33. The mutant of claim 31, having a truncated C-terminal regulatory region.

34. The mutant of claim 31, having a mutation in the phosphorylation recognition region and a truncated C-terminal regulatory domain.

35. The mutant of claim 32, having a mutation in an amino acid corresponding to any one of Arg171, Arg175, Lys 167 and Lys176 of SEQ ID NO: 4.

36. The mutant of claim 35, having a mutation in an amino acid corresponding to Arg175 of SEQ ID NO: 4.

37. A phosphorylation-independent β arrestin mutant having the amino acid of SEQ ID NO: 7.

38. A phosphorylation-independent β arrestin mutant having the amino acid sequence of SEQ ID NO: 8.


SEQUENCE LISTING (8 sequences)

SEQ ID NO: 1 - protein, 418 aa, Bos taurus
MGDKGTRVFKKASPNGKLTVYLGKRDFVDHIDLVEPVDGVVLVDPEYLKERRVYVTLTCAFRYGREDLDVLGLTFRKDLFVANVQSFPPAPEDKKPLTRLQERLIKKLGEHAYPFTFEIPPNLPCSVTLQPGPEDTGKACGVDYEVKAFCAENLEEKIHKRNSVRLVIRKVQYAPERPGPQPTAETTRQFLMSDKPLHLEASLDKEIYYHGEPISVNVHVTNNTNKTVKKIKISVRQYADICLFNTAQYKCPVAMEEADDTVAPSSTFCKVYTLTPFLANNREKRGLALDGKLKHEDTNLASSTLLREGANREILGIIVSYKVKVKLVVSRGGLLGDLASSDVAVELPFTLMHPKPKEEPPHREVPEHETPVDTNLIELDTNDDDIVFEDFARQRLKGMKDDKEEEEDGTGSPRLNDR

SEQ ID NO: 2 - protein, 418 aa, Homo sapiens [sequence omitted]

SEQ ID NO: 3 - protein, 410 aa, Homo sapiens [sequence omitted]

SEQ ID NO: 4 - protein, 409 aa, Homo sapiens
MGEKPGTRVFKKSSPNCKLTVYLGKRDFVDHLDKVDPVDGVVLVDPDYLKDRKVFVTLTCAFRYGREDLDVLGLSFRKDLFIATYQAFPPVPNPPRPPTRLQDRLLRKLGQHAHPFFFTIPQNLPCSVTLQPGPEDTGKACGVDFEIRAFCAKSLEEKSHKRNSVRLVIRKVQFAPEKPGPQPSAETTRHFLMSDRSLHLEASLDKELYYHGEPLNVNVHVTNNSTKTVKKIKVSVRQYADICLFSTAQYKCPVAQLEQDDQVSPSSTFCKVYTITPLLSDNREKRGLALDGKLKHEDTNLASSTIVKEGANKEVLGILVSYRVKVKLVVSRGGDVSVELPFVLMHPKPHDHIPLPRPQSAAPETDVPVDTNLIEFDTNYATDDDIVFEDFARLRLKGMKDDDYDDQLC

SEQ ID NO: 5 - protein, 388 aa, Homo sapiens [sequence omitted]

SEQ ID NO: 6 - protein, 388 aa, Homo sapiens
MSKVFKKTSSNGKLSIYLGKRDFVDHVDTVEPIDGVVLVDPEYLKCRKLFVMLTCAFRYGRDDLEVIGLTFRKDLYVQTLQVVPAESSSPQGALTVLQERLLHKLGDNAYPFTLQMVTNLPCSVTLQPGPEDAGKPCGIDFEVKSFCAENPEETVSKRDYVRLVVRKVQFAPPEAGPGPSAQTIRRFLLSAQPLQLQAWMDREVHYHGEPISVNVSINNCTNKVIKKIKISVDQITDVVLYSLDKYTKTVFIQEFTETVAANSSFSQSFAVTPILAASCQKRGLALDGKLKHEDTNLASSTIIRPGMDKELLGILVSYKVRVNLMVSCGGILGDLTASDVGVELPLVLIHPKPSHEAASSEDIVIEEFTRKGEEESQKAVEAEGDEGS

SEQ ID NO: 7 - protein, 409 aa, Artificial Sequence
  Description of Artificial Sequence Homo sapiens mutation
MGEKPGTRVFKKSSPNCKLTVYLGKRDFVDHLDKVDPVDGVVLVDPDYLKDRKVFVTLTCAFRYGREDLDVLGLSFRKDLFIATYQAFPPVPNPPRPPTRLQDRLLRKLGQHAHPFFFTIPQNLPCSVTLQPGPEDTGKACGVDFEIRAFCAKSLEEKSHKRNSVRLVIEKVQFAPEKPGPQPSAETTRHFLMSDRSLHLEASLDKELYYHGEPLNVNVHVTNNSTKTVKKIKVSVRQYADICLFSTAQYKCPVAQLEQDDQVSPSSTFCKVYTITPLLSDNREKRGLALDGKLKHEDTNLASSTIVKEGANKEVLGILVSYRVKVKLVVSRGGDVSVELPFVLMHPKPHDHIPLPRPQSAAPETDVPVDTNLIEFDTNYATDDDIVFEDFARLRLKGMKDDDYDDQLC

SEQ ID NO: 8 - protein, 382 aa, Artificial Sequence
  Description of Artificial Sequence Homo sapiens mutation
MGEKPGTRVFKKSSPNCKLTVYLGKRDFVDHLDKVDPVDGVVLVDPDYLKDRKVFVTLTCAFRYGREDLDVLGLSFRKDLFIATYQAFPPVPNPPRPPTRLQDRLLRKLGQHAHPFFFTIPQNLPCSVTLQPGPEDTGKACGVDFEIRAFCAKSLEEKSHKRNSVRLVIRKVQFAPEKPGPQPSAETTRHFLMSDRSLHLEASLDKELYYHGEPLNVNVHVTNNSTKTVKKIKVSVRQYADICLFSTAQYKCPVAQLEQDDQVSPSSTFCKVYTITPLLSDNREKRGLALDGKLKHEDTNLASSTIVKEGANKEVLGILVSYRVKVKLVVSRGGDVSVELPFVLMHPKPHDHIPLPRPQSAAPETDVPVDTNLIEFDTNYAT